(19) United States
(12) Patent Application Publication
(10) Pub. No.: US 2005/0250974 A1
(43) Pub. Date: Nov. 10, 2005

(54) Therapeutic handbags and carrying bags

(76) Inventors: Lauren Elizabeth Bailey, Alexandria, VA (US); Alison Paige Corday, Landenberg, PA (US); Stephanie Facciolo, Wilmington, DE (US)

Correspondence Address: MARK G. MORTENSON, POST OFFICE BOX 310, NORTH EAST, MD, 21901-0310, US

(21) Appl. No.: 11/108,563

(22) Filed: Apr. 18, 2005

Related U.S. Application Data
(60) Provisional application No. 60/563,498, filed on Apr. 19, 2004.

(51) Int. Cl. A61N 2/00; A61B 17/52; A61N 1/00; A45C 1/02; A45C 3/06

(52) U.S. Cl. 600/15; 600/9; 150/100

(57) ABSTRACT

The present invention relates to handbags and carrying bags of various types used or carried by men and women. More particularly, the present invention relates to handbags or carrying bags that produce therapeutic benefits for the user. Even more particularly, the present invention relates to handbags or carrying bags containing or including at least one therapeutic device therein. Therapeutic devices include at least one magnet or magnetic device, as well as certain device(s) that produce sympathetic resonances.

This application claims the benefit of U.S. Provisional Application No. 60/563,498, filed Apr. 19, 2004.

### FIELD OF THE INVENTION

The present invention relates to handbags and carrying bags of various types used or carried by men and women. More particularly, the present invention relates to handbags or carrying bags that produce therapeutic benefits for the user. Even more particularly, the present invention relates to handbags or carrying bags containing or including at least one therapeutic device therein. Therapeutic devices include at least one magnet or magnetic device, as well as certain device(s) that produce sympathetic resonances.

### BACKGROUND OF THE INVENTION

Various attempts have been made to provide therapeutic pads or devices which rely on the healing capabilities of magnets. For example, devices which utilize magnetic alternating polarities to increase blood flow and therefore, accelerate healing of injured body parts, are known. Principles of electricity and magnetism set forth In Faraday's Law of Magnetic Induction and the Hall Effect may come into play in magnetic therapy. For example, charged particles experience a force acting upon them when they move through a magnetic field in a perpendicular direction. Human blood contains ions and electrolytes and may thus be acted upon by one or more magnetic fields. Thus, for example, a blood vessel exposed to a magnetic field could experience a widening of the blood vessel and thus an increase in blood flow. Such increased blood flow may result in enhanced healing and/or less stress.

Studies conducted by Ron Lawrence M.D., Ph.D., the President of the Academy of Magnetic Therapy, measured a 300% increase in blood flow when the blood vessels were exposed to a magnetic field. These measurements were determined via testing by plethysmography, a way of measuring blood flow through the fingers.

The increase of blood flow also speeds the exchange of fluids such as lactic acid. The exchange of these fluids in injured tissue further reduces pain. Magnetic therapy can also heal more severe pain found in human bones. The FDA has approved the use of electromagnetic therapy to treat fractured bones that have failed to heal normally. In addition, studies conducted by Drs. Robert Becker and Andrew Bassett also show the positive affects of magnets on regeneration and the healing process in both humans and animals.

Permanent magnets have been used to treat the legs of racehorses for many years. The effect of magnets on humans is much the same with animals. The use of magnets are harmless to both animals and humans. The work of The National Institute of Health has shown that the Magnetic Resonance Imaging's (MRI) strong magnetic fields have no harmful effects.

Magnets have been shown to calm children with Attention Deficit Disorder, reduce headaches, lessen the pain associated with fybromyalgia and post-polio syndrome, and can reduce insomnia. Studies by Dr. Michael I. Weintraub of the New York Medical College Department of Neurology and Medicine showed the positive effects of electromagnetic therapy on diabetes patients and those suffering from Carpal Tunnel Syndrome. In addition, the relaxation effects of electromagnetic therapy can also prevent asthma and reduce insomnia. Magnets can also heal by creating a balance in the human body.

Studies have proven that the Earth's magnetic fields play a large role in the health of its inhabitants. In the United States, numerous scientific journals have reported the beneficial therapeutic effects of magnets since the 1970's, and the FDA has approved the use of pulsed electromagnetic fields for the treatment of nonunion bone fractures.

It is also known by NASA that exposure to the Earth's magnetic fields seems to play an essential role to human health, a fact demonstrated when the first astronauts returned to Earth sick. Their illness was soon attributed, at least partially, to the lack of magnetism in outer space and the problem was subsequently resolved when NASA placed magnets in their space suits and spaceships. Since, it has been discovered that in the absence of a magnetic field, the energy level of atoms may diminish (or at least change), required nutrients may become depolarized and unusable. If this condition is permitted to continue, the body can become imbalanced and function improperly. By restoring balance to an organism, biomagnetic therapy can alleviate a number of health conditions.

Basically, to understand how a magnet helps alleviate pain, it is important to understand how pain works in the body. Pain is transmitted along nerves cells in the form of an electric signal. This pain signal depolarizes the cell. Magnets may block the pain by raising the polarization of the cell. Also, the ability of the nerve to send pain appears to be slowed by a magnetic field. Accordingly, by understanding this process, it is possible to aid pain relief throughout the body.

With regard to injured tissue, magnets help as well. A positive charge is emitted when tissue is injured. Placing the negative pole of a magnet over the injured area appears to restore a natural balance by improving circulation, allowing blood vessels to dilate and bringing a greater volume of blood flow to the injured area.

Another important healing component of magnets their apparent ability to ward off microbial assailants such as viruses, bacteria, and fungi. Magnets have the ability to increase immune functions by, for example, oxygenating white corpuscles, an important part of the immune system. Magnets may thus achieve an antibiotic function by lowering acidity, with the result that certain harmful organisms have a more difficult time surviving. The net effect is to augment the body's natural ability to resist a variety of germs.

In terms of stress, anxiety and depression reduction, magnets appear to result in certain calming effects as well. With the recent discovery of magnetite in the brain, it is certainly possible that magnetic fields can have a positive effect on the state of the brain.

Also, in terms of energy enhancement, it is believed that magnetic energy has the ability to penetrate all facets of the human body and reach every cell. A healthy cell, (e.g., a polarized cell) includes a positively charged nucleus and a negatively charged outer membrane. However, as a cell performs its daily functions, it becomes depolarized. Depolarized cells are not optimum for peak performance and may result in a weakened or tired state. One normally revitalizes energy during sleep. Therefore magnetic fields can polarize the cells, potentially re-energizing the entire body.

The aforementioned ailments of stress and low energy levels, once relieved by magnetic energy, can drastically reduce the effects of aging. In addition, magnets appear to activate life-promoting enzymatic activity that encourages normal cell division. This probably creates a healthier organism and may slow down the aging process. Consequently, both mental and physical benefits result from magnetic therapy and thus translate into a greater vitality and well being, ultimately improving the quality of life, and possibly lengthening life span.

Several exemplary U.S. Patents address the use of magnets. For example, U.S. Pat. No. 4,489,711 to Latzke, discloses alternating stripes of north and south magnetic poles that are unidirectional in pole orientation. Latzke '711 provides parallel strip-shaped poles in a linear arrangement that do not exhibit therapeutic effects on blood vessels that are not extending substantially transversely thereto.

Magnetic therapy has resulted in the development of products containing permanent magnets. For example, U.S. Pat. No. 4,509,219 teaches a sleeping mattress structure provided with permanent magnets each having a magnetic field strength of at least 850 gauss which are disposed on the mattress for maximum magnetic curing effects.

U.S. Pat. No. 4,921,560 teaches a method for fixing the permanent magnets to bed covering.

Merchandise worn by humans having various magnetic structures has also been developed. For example, the Nikken Wellness U.S. Product Reference Guide, Pages 18-21, 2002 edition, shows humanwear merchandise provided with magnetic structure including headbands, vests, belts, wrist bands, supports for the elbows, arms, legs, knee and ankle, and also necklaces.

Additionally, magnets have been placed in wraps, jewelry and the soles of shoes to provide therapeutic effects. Magnets have also been incorporated into numerous other devices, such as those described in U.S. Pat. No. 4,480,596 (lumbar belts), U.S. Pat. No. 5,642,739 (armbands), and U.S. Pat. No. 5,720,046 (clothing). Each device is used for exposing a specifically defined, external region of the human body to a magnetic field.

Further, it has been reported that biomagnets can be placed at strategic points on the body to positively influence pain control by directing magnetic fields to acupuncture points, acupuncture meridians, and the surrounding hard and soft tissue. For example, clothing having discrete magnets attached thereto for producing healing effects at acupuncture points has been disclosed in U.S. Pat. No. 5,720,046, which issued Feb. 24, 1998, to Lopez et al. More specifically, Lopez teaches clothing and other personal wear which utilizes discrete magnets to direct magnetic fields towards acupuncture points along the human torso; acupuncture points on the head along the temporal region, soft spot and upper most vertebrae region at the base of the skull; and acupuncture points on the body appendages.

Thus, merchandise provided with a magnetic structure (e.g., a permanent magnet) causes body cells to be exposed to a low-level magnetic field emitted from the permanent magnets. The magnetic exposure is believed to assist stressed cells in restoring their correct balance of electrical charge for performing more efficiently. The permanent magnets are commercially available in a variety of shapes and magnetic strengths which produce a variety of discrete fields, many of which are now known to produce therapeutic effects. One approach by the user of the discrete permanent magnets has been to tape the magnet onto the particular body point being treated or use one of the known magnet-carrying devices which applies the magnetic field to the body.

Also known are devices which utilize something known as Sympathetic Resonance Technologies™ (SRT™). Devices utilizing this technology are marketed by Clarus Products International L.L.C. and are known as the “Clear Wave” (an alarm clock), the “Ally” (a battery driven device) and the “Q-link” (a passive pendant). These devices are also known to provide therapeutic effects by, for example, utilizing subtle energies to affect chemical, physical and/or biological systems. The SRT™ devices claim to boost one's overall mental, emotional and physical energy states at the most fundamental level of life energy. However, these types of devices use natural energies and/or subtle energies to produce a positive energy, extremely beneficial to the user of such objects. Moreover, biological and clinical studies collectively show that SRT™ devices lessen the stress response for a variety of stressors such as chemical and electromagnetic stress. SRT™ devices may stabilize the organism in many ways, thus, protecting the organism from the effects of stressful stimuli and thereby enhancing the healing response and supporting general wellness. The Q-link has gained wide acceptance among many professional golfers.

The various known magnetic devices and/or SRT ™ devices produce various positive therapeutic or physiological effects, however, the prior art devices do not contemplate a delivery vehicle which maximizes the desirable effects of such devices when such effects are needed the most. The present invention fills this unsatisfied need.

### SUMMARY OF THE INVENTION

The present invention relates in general to handbags and carrying bags, and in particular to a new and useful bags that exhibit therapeutic effects. Specifically, the present invention provides a novel delivery vehicle for delivering therapeutic treatment just when such treatment may be needed the most.

Most men and women need multiple items to support their daily activities. These items include credit cards, photographs, credentials, glasses, a checkbook, a wallet, change purse, money, keys, stamps, note paper, phone number listing, facial tissues, pharmaceuticals, cosmetics, etc. These items are typically used in an environment which involves physical and/or mental stresses such as in an automobile, travelling by various commercial modes of transportation, shopping, school, business settings, sports or physical activities, etc.

To accommodate these needs, the prior art is replete with handbags or carrying bags of various sizes, shapes and designs. Many such handbags are quire fashionable as well as functional. In many cases, the handbags can be quite heavy. Thus, when a handbag strap is carried or placed over a shoulder, certain stresses can be exerted on the body. Likewise, when a handbag strap is placed one's hand, stresses are exerted on the hand. Thus, physical stress can result in all areas of the arms and shoulders (including the elbows and fingers) as well as the neck. Fibromyalgia is the only disorder more prevalent than the common cold. The condition is characterized by varying degrees of discomfort and pain in the muscles. Many people that have Fibromyalgia suffer from pain in the shoulder and neck, especially women. Therapeutic handbags serve to combat those stresses. For example, magnets and/or other therapeutic technologies that are strategically placed in the handbag and/or throughout the strap in the handbag can reduce the amount of pain and discomfort experienced by the user. As discussed above, magnets work to reduce pain by, for example, effecting the human blood stream.

Further, devices such as the Q-link, Ally and Clear Wave, have been developed by Clarus Products International L.L.C. (as discussed above). These devices use a technology called ‘Sympathetic Resonance Technology™’ (SRT™) that affect chemical, physical and biological systems. Accordingly, the present invention contemplates using such devices alone or in combination with magnets in the therapeutic bags. For example, by using the combination of at least one magnet and/or at least one SRT™ device in a bag, the overall well being of a person who carries such a bag could be amplified in several ways, unique to the delivery vehicle according to the present invention.

For example, when individuals are exposed to the rigors of everyday life, mental stress, in addition to physical stress, can take a heavy toll on various physiological functioning of a body. By providing a delivery system which is present during stressful events, as well as by providing therapeutic delivery to portions of a body that need it the most, the health and well-being of individuals may be promoted.

For example, the usage of magnets in bags would help the user in many ways. Proper placement of magnets in, for example, the straps of a standard bag, could help relieve shoulder pain. Depending on the bag, back pain, hand/wrist pain, waist pain, chest pain and arm pain, could all be aided by the power of appropriately positioned magnets at those points where a body experiences the greatest physical stress. In the same vein, reduction of inflammation, improved circulation, arthritis pain, and antimicrobial effects could be alleviated when magnets are suitably incorporated into bags.

Further, usage of magnets and/or SRT™ devices in the delivery vehicle according to the present invention can produce enhanced reduction of stress, anxiety and depression, enhancement of energy and anti-aging effects as well. For those individuals that have a serious interest in gaining a balanced health and overall well being, a bag that incorporates at least one magnet and/or at least one SRTTM device would be ideal. Furthermore, it has been observed that a non-continuous use of magnets or the use of magnets at intervals may be more beneficial than a continuous use. Accordingly, the placement of such devices into at least a portion of a handbag, and/or a handbag strap, could be very beneficial because handbags are not continuously used. Accordingly, carrying a bag off and on all day and night, (i.e., at intervals) would be perfect for these desired therapeutic effects.

Further, SRT™ devices which utilize sympathetic resonance technologies to, for example, shield the body from electromagnetic radiation, could also be incorporated into at least a portion of the handbag, with or without additional magnetic devices present therein. For example, the devices discussed above and sold under the name Clear Wave, Ally and Q-Link (all marketed by Clarus Products International L.L.C.) have been shown by clinical trails to: strengthen resilience and resistance to the effects of stress; increase energy and enhance mental performance, especially under pressure; and boost capacity to function within electromagnetic field saturated environments.

Undesirable electromagnetic radiation is found in many parts of the environment as well as in many products often carried in handbags and totes. These products include but are not limited to cell phones, beepers and computers. Moreover, many devices create electromagnetic radiation in the environment including, but not limited to, TV sets, radar transmitters, transformers, power lines, etc. Electromagnetic radiation itself, can cause fatigue, headaches, increased blood pressure, and many studies have linked exposure to electromagnetic radiation to cancer, Alzheimer's and Parkinson's Disease (it is noted that debate still exists in these fields).

The purpose of placing the SRT™ devices in handbags and totes would be to reduce the amount of harmful physiological effects due to exposure, to for example, electromagnetic radiation in the environment.

Accordingly, an object of the present invention is to provide a handbag which provides therapeutic treatment for the user. In a first embodiment of the invention, therapeutic treatment is accomplished by providing a handbag that contains at least one magnetic structure for affecting therapeutic magnetic exposure when used by a human. In particular, by incorporating at least one magnetic structure in, for example, the sides of the handbag that contact a human body; the strap that contacts the human hand; or the sides and/or strap that contacts the human torso, the body may experience beneficial magnetic exposure.

The surface provided by the side of a handbag can result in the potential for a large surface area of the body being exposed to a beneficial magnetic field. This is to be contrasted with the small magnetic field that may result from, for example, magnetic closure devices used to hold some handbags shut. Moreover, the present invention contemplates the use of, for example, magnets that have been specifically shown to achieve desirable physiological effects (e.g., those marketed by Nikken and known as EQL magnetic and FAR-infrared technologies, KENKO™ magnetic pads and/or Elastomag® wraps, rather than simple permanent magnets known to the art for their uses. In other words, certain magnets have been shown to produce enhanced positive biological results relative to others.

Further, by incorporating at least one magnet in a strap portion of a handbag, a human hand and/or a human torso may benefit from magnetic exposure. In this regard, the incorporation of a suitable physiological magnet into the strap of a bag could provide unique magnetic field exposure to the torso of an individual. For example, if a strap had an extended length and was attached to a bag such that the strap crossed a large portion of a torso (e.g., diagonally form left shoulder to right waist) and the strap contained desirable physiological magnets (e.g., such as those discussed above) throughout greater than 25% of its length, or in a preferred embodiment greater than 50% of its length, or in a most preferred embodiment, through at least 90% of its length, then relatively extensive magnetic field exposure will result compared to other portable magnets and/or SRT™ device technologies known to the art.

Specifically, almost all vital organs in the body can be exposed to a desirable magnetic field created by magnets contained in a strap that diagonally crosses the body. Never before has such a desirable field delivery system been contemplated that is portable and inconspicuous. Moreover, the added weight due to such magnets is very minimal due to the current technologies available for creating physiologically beneficial magnets.

Further, additional devices which utilize sympathetic resonance technologies to, for example, shield the body from electromagnetic radiation, could also be incorporated into at least a portion of the handbag. For example, devices sold under the name Clear Wave, Ally and Q-link (all marketed by Clarus Products International L.L.C.) are known to: strengthen resilience and resistance to the effects of stress; increase energy and enhance mental performance, especially under pressure; and boost capacity to function within electromagnetic field saturated environments. These devices utilize a technology that is referred to as Sympathetic Resonance Technology. Any device exhibiting this technology, when incorporated into a handbag, could provide desirable therapeutic effects. Specifically these SRT™ devices could be positioned or located in bags similar to the aforementioned magnetic devices. Accordingly, such SRT™ devices could be made to be larger in size, if desired.

Thus, the placement of one or more magnets and/or SMT™ devices in a strap portion of a bag, and/or in a bag itself, could result in a large portion of the torso of a human body being exposed to these therapeutic effects. No devices known in the art have a capacity for exposing such large portions of a human body (e.g., a human torso) to such desirable effects precisely when such effects are most needed.

### BRIEF DESCRIPTION OF THE FIGURES

FIG. 1 shows a typical handbag in perspective view.

FIG. 2 shows a typical handbag with a longer strap in perspective view.

FIG. 3 shows a fanny pack.

FIG. 4 shows a typical handbag with inserts shown in cross-hatching.

FIG. 5 shows a typical handbag with inserts shown in cross-hatching.

FIG. 6 shows a typical handbag with inserts shown in cross-hatching.

FIG. 7 shows a typical handbag with inserts shown in cross-hatching.

FIG. 8 shows a fanny pack with inserts shown in cross-hatching.

FIG. 9 shows a perspective view of a fanny pack on a user.

FIG. 10 shows a perspective view of a briefcase/suitcase in the hand of a user.

FIG. 11 shows a front perspective view of a strap attached to a laptop bag, mailbag, newspaper bag, purse, etc., diagonally fitting across the front of a user.

FIG. 12 shows a back perspective view of a strap attached to a laptop bag, mailbag, newspaper bag, purse, etc., diagonally fitting across the back of a user.

FIG. 13 shows a perspective view of the strap of a tote around the shoulder of a user.

FIG. 14 shows a perspective view of a handbag or purse around the shoulder of a user.

FIG. 15 shows a partial perspective view of a clutch in the hand of a user.

FIG. 16 shows a partial perspective view of a clutch in the armpit of a user.

FIG. 17 shows a perspective view of a handbag on the lap of a seated user.

FIG. 18 shows a perspective view of a handbag next to the leg of a seated user.

FIG. 19 shows a perspective view of a handbag in the hand of a user.

FIG. 20 shows a front perspective view of the straps of a backpack on a user.

FIG. 21 shows a back perspective view of a backpack on a user.

FIG. 22 shows a front perspective view of a strap diagonally across a user, the strap being attached to a golf bag.

FIG. 23 shows a back perspective view of a golf bag diagonally across a user, the strap being attached to a golf bag.

### DETAILED DESCRIPTION OF THE INVENTION

The present invention provides a unique delivery device for creating/delivering desirable fields to the body. In particular, the present invention contemplates the use of one or more magnetic structures to provide therapeutic treatment to the human body. The magnets may be placed in the sides, bottom, pockets or across the straps, in any desirable and physiologically suitable arrangement. The bag may also contain flexible or somewhat rigid magnetic plates such as the Kenko™ Magnetic Promo Pad sold by Nikken, EQL magnetic and FAR-infrared technology products, KENKO™ magnetic pads and/or Elastomag® wraps, rather than simple permanent magnets known to the art for their uses. The magnetic plates and other magnetic materials may also be used as functional closures, however, the plates of the present invention should be significantly larger than the size required to merely close the opening of a bag, and/or be located in other portions of the bag, in order to provide the desired therapeutic effects according to the novel delivery device of the present invention. The handbag may also be constructed with magnetic fibers. In addition, the lining and other areas of the bag may be constructed with a series of magnets. The placement and number of each of these magnetic devices are not restricted to any part of the handbag. However, the placement of the magnets should be such that the created fields interact favorably with targeted areas of the anatomy. In this regard, whenever physical stress is created by carrying the bag, magnets should be positioned to minimize physical stresses. Further, the magnets can be located so as to be contiguous to large portions of a human's anatomy to maximize desirable effects.

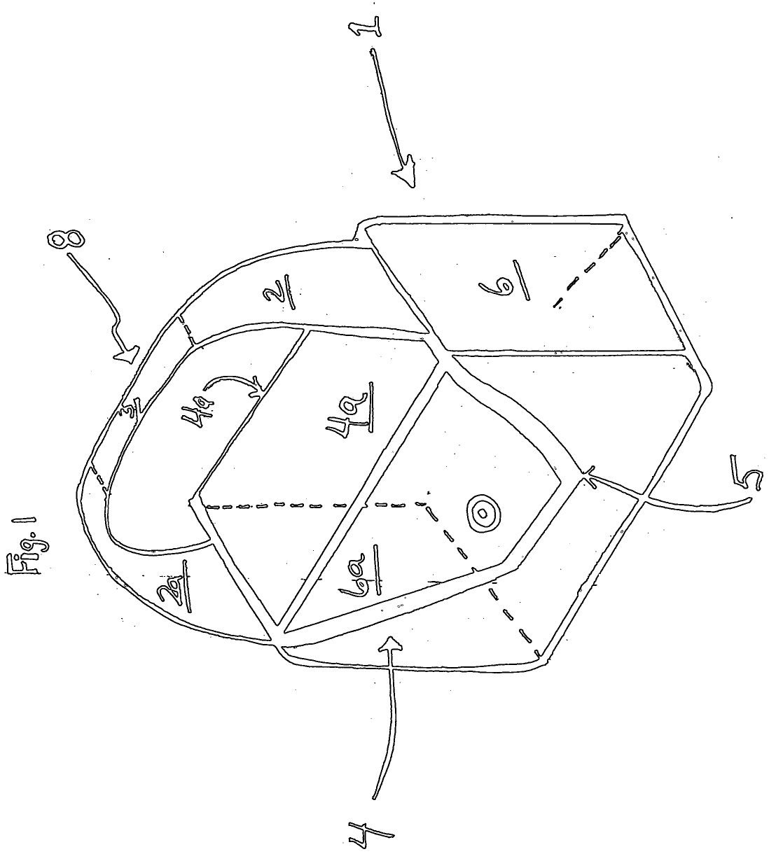

FIG. 1 shows a typical handbag having a carrying portion for carrying various articles comprised of a front side 4, a back side 4a, one end 6 and an opposite end 6a. The carrying portion is attached to a strap portion 8 which has a section 2 and complimentary section 2a as well as a section 3. The section 3 is the area which a hand would typically grasp the strap 8. A flap 5 serves to close the handbag 1. The portions 2 and 2a of the strap 8 typically do not contact a user's hand when carrying the strap 8.

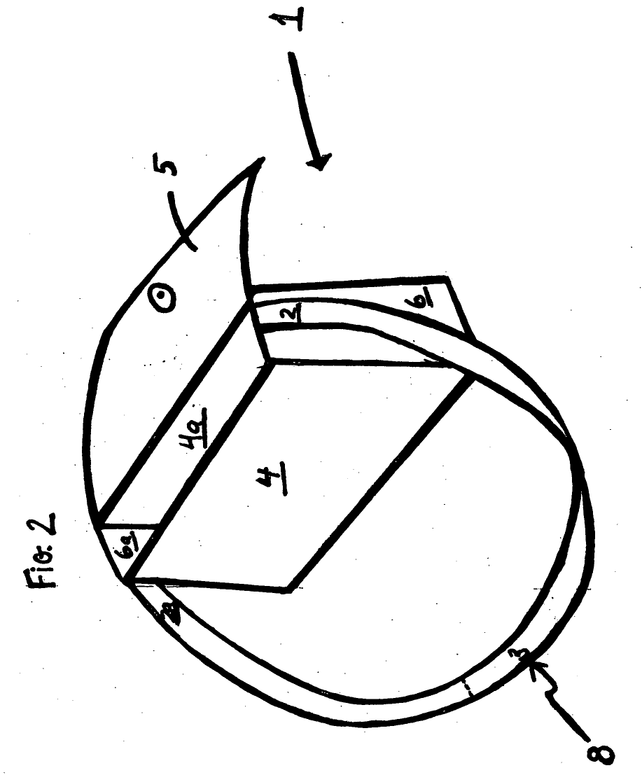

A similar handbag is shown in FIG. 2. However, the primary differences between the handbag of FIG. 2 and the handbag of FIG. 1 is that a longer strap portion 8 is provided for the handbag 1 shown in FIG. 2. This type of handbag would typically refer to the strap portion 8 as a shoulder strap. Often such a shoulder strap is draped across the body in a diagonal manner, as well as hanging from a single shoulder substantially straight down.

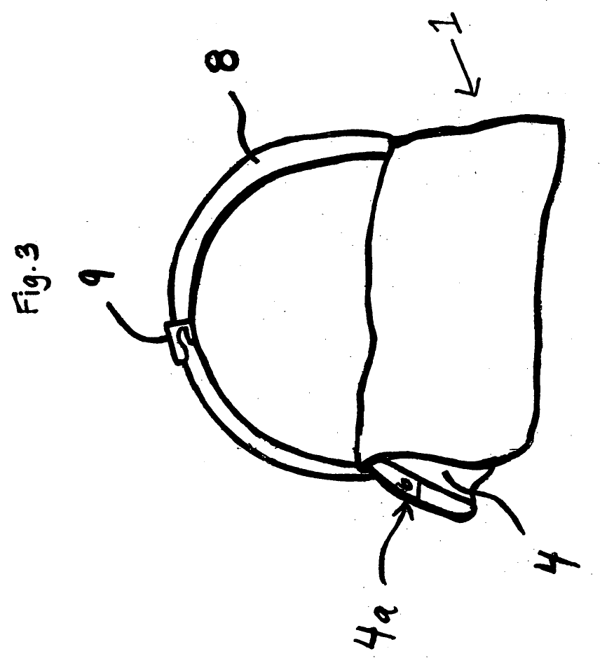

A typical fanny pack is shown in FIG. 3. In this figure, the portion 4a would be juxtaposed against the back (or front) of an individual and the strap 8 comprises a belt portion with a buckle 9.

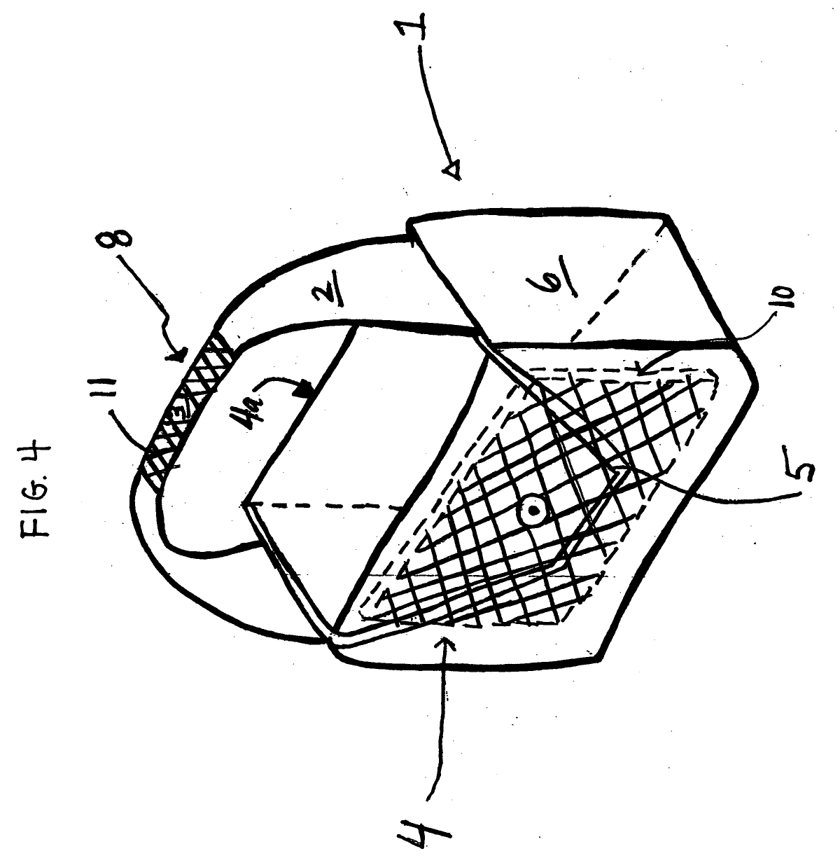

FIG. 4 shows a handbag according to the present invention. In this FIG. 4, the crosshatched portions 10 and 11 correspond to, for example, a magnetic material. For example, the magnetic material may comprise a substantially rigid or a somewhat flexible magnetic sheet material. A suitable magnetic material includes, for example, the Kenko™ Magnetic Flex Pad. Likewise, a crosshatched portion 11 is shown in the strap portion 8 of the handbag 1. Of course, one or both of the sections 10 and 11 could be provided in a single handbag.

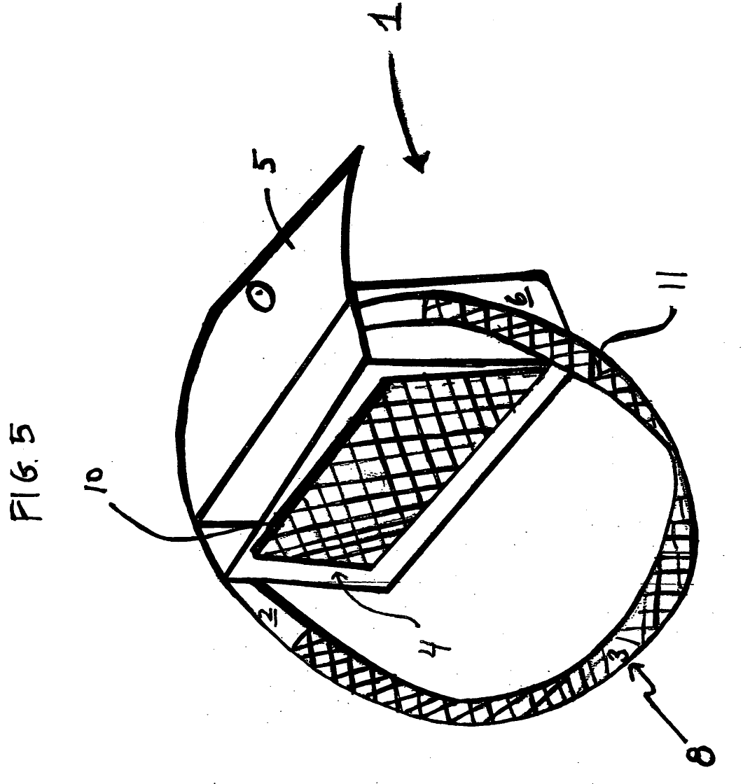

The handbag 1 shown in FIG. 5 contains similar portions 10 and 11 to those shown in FIG. 4. However, the section 11 in the strap portion 8 is much larger because the strap 8 is designed to be hung from a single shoulder or diagonally across the body. Likewise, in this figure, the portions 10 and 11 could be provided separately.

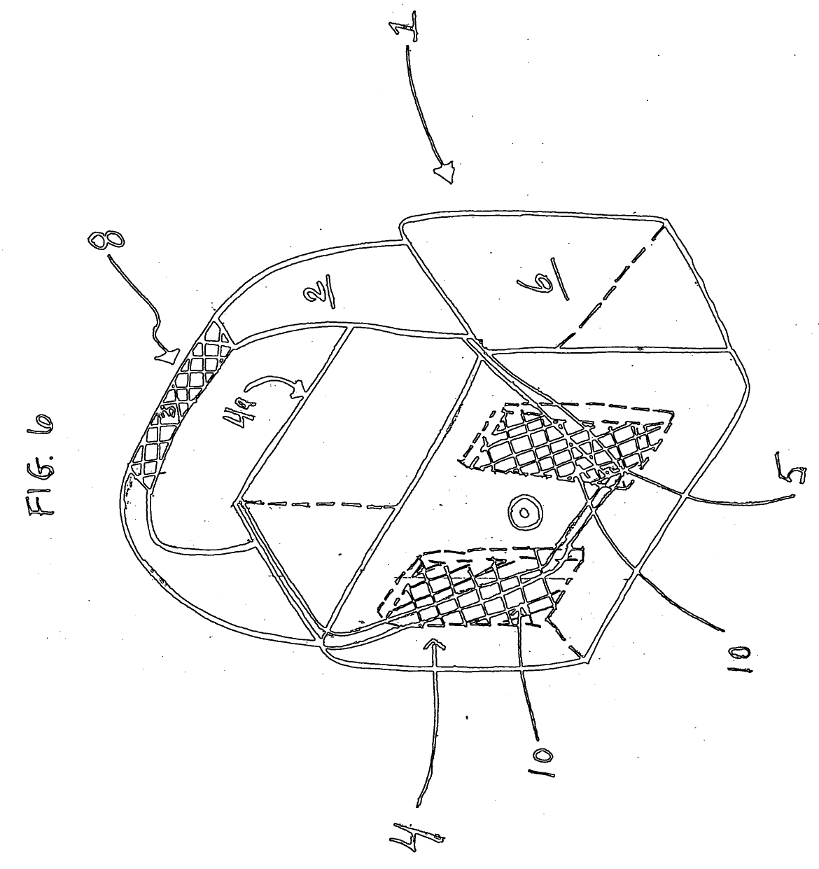

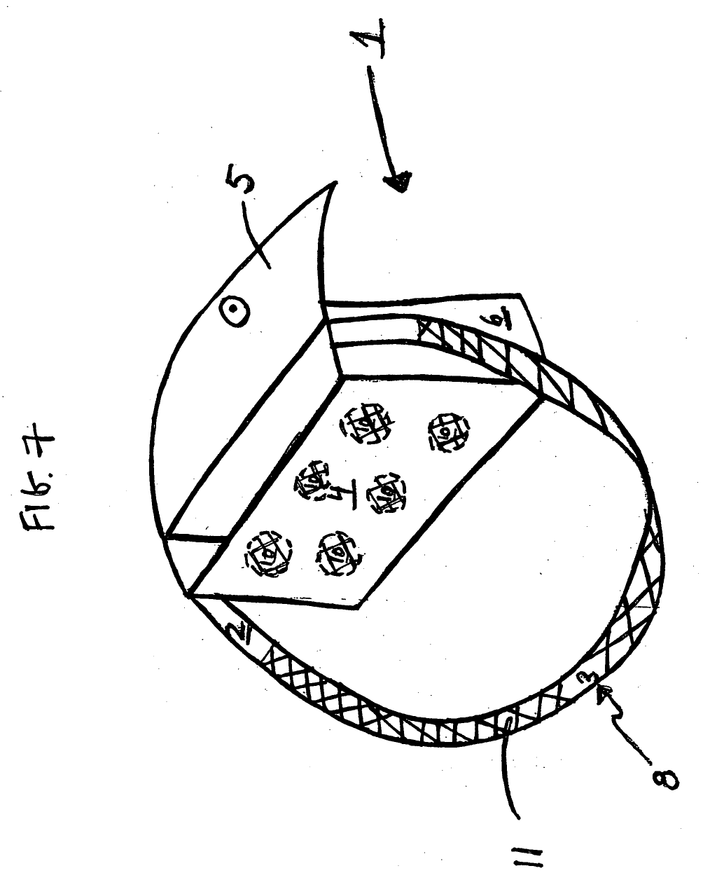

FIGS. 6 and 7 show alternative configurations to those shown in FIGS. 4 and 5, respectively. It should be understood that the inserts shown in the handbags could be of any particular convenient size and/or shape as well as number. Such considerations include flexibility of the handbag due to the inserts 10 and 11 provided, functionality of the handbags due to the inserts provided, etc.

Still further, in another embodiment of the invention, rather than providing a magnetic material in those portions 10 and 11 shown in FIGS. 4-6, one or more sympathetic resonance technology devices could be provided. In this regard, an active device, such as an Ally or a passive device similar to a device known as the Q-link pendant could be provided. Of course, the specific designs of such inserts would, like for magnetic inserts, be dependent on functionality of not only the handbag, but the functionality of the device. In this regard, it should be understood that the close proximity of any inserts, whether magnetic or otherwise, will provide more desirable effects when located in a juxtaposed position to the body (e.g., either the hand, shoulder, torso, or in cases where handbags are held even portions of the upper thighs or hip regions of a human body).

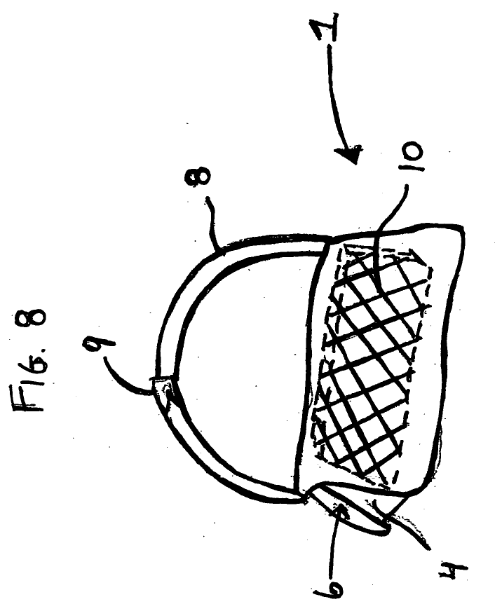

FIG. 8 shows a typical fanny pack which also contains a portion 10. This portion 10 is similar to the portions 10 discussed FIG. 4-7 immediately above. Likewise, the strap portion 8 could also contain similar magnetic inserts and/or sympathetic resonance technology inserts.

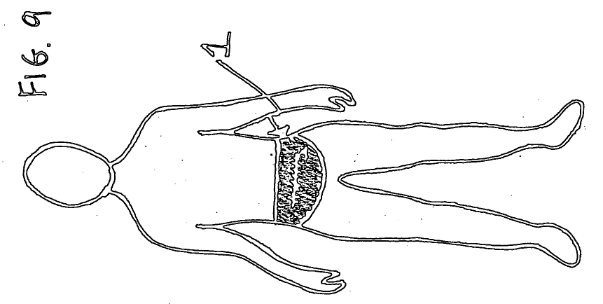

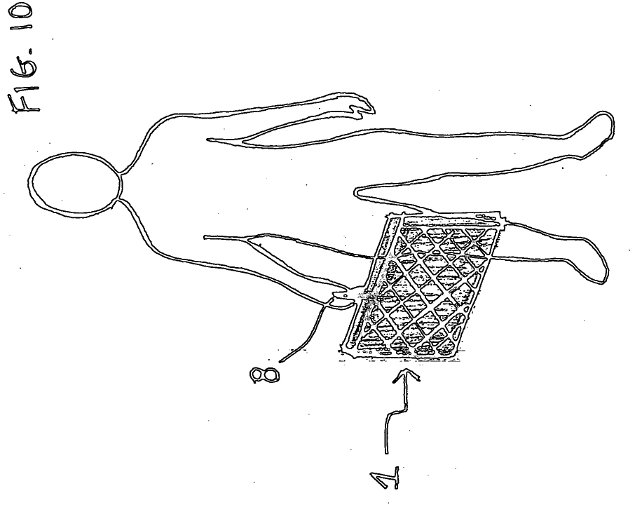

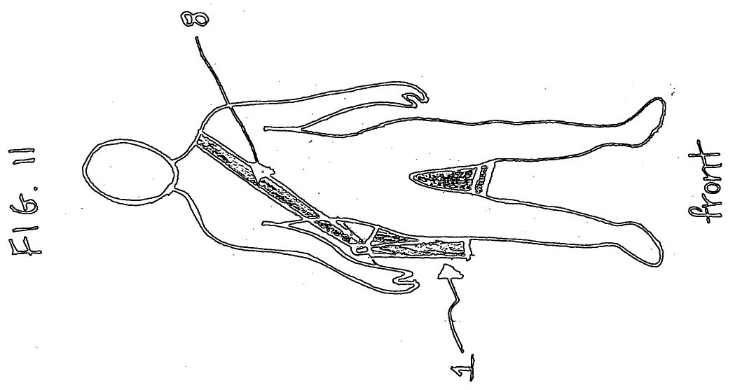

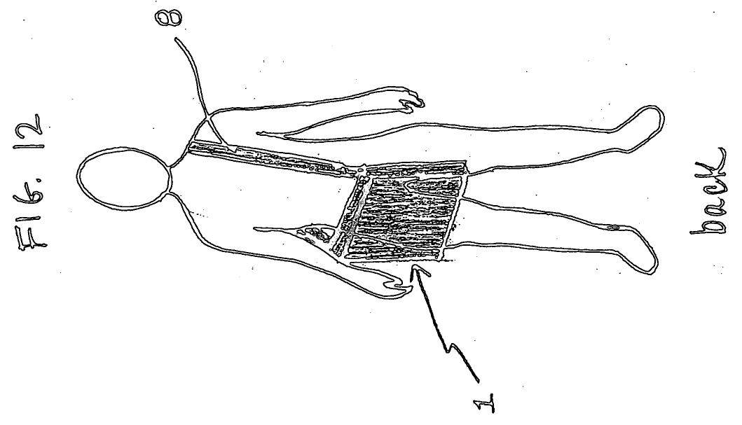

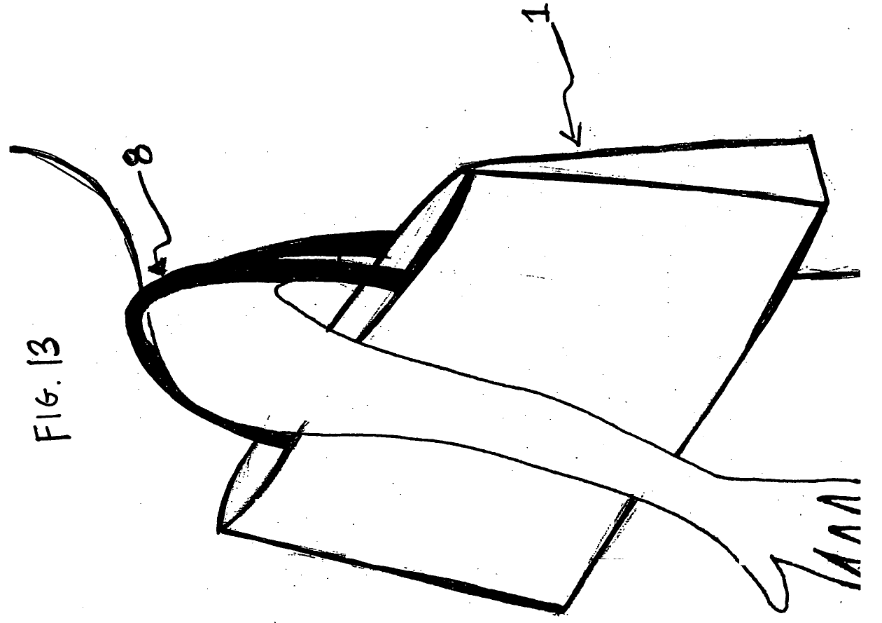

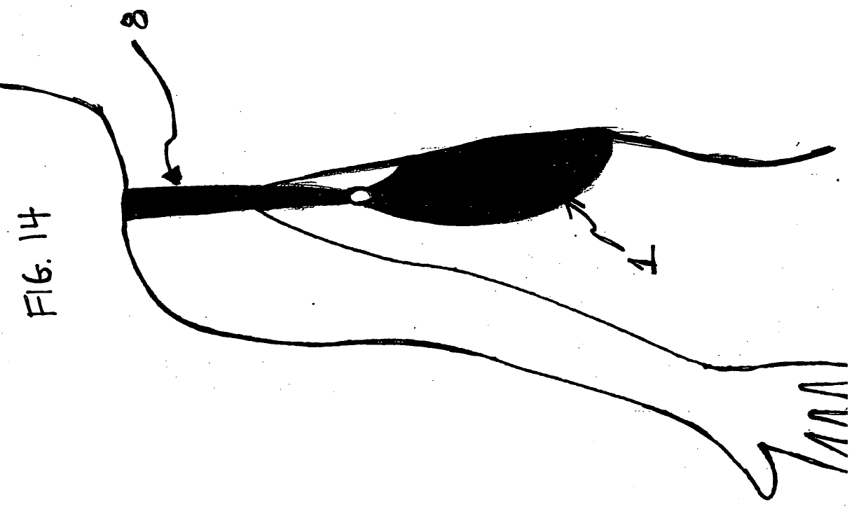

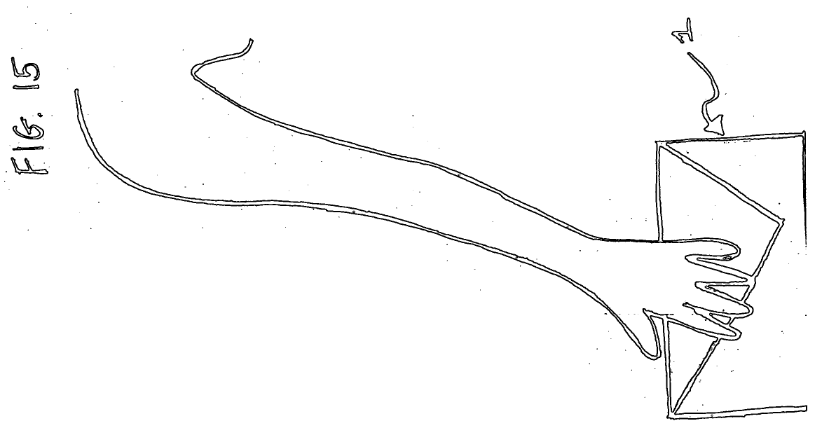

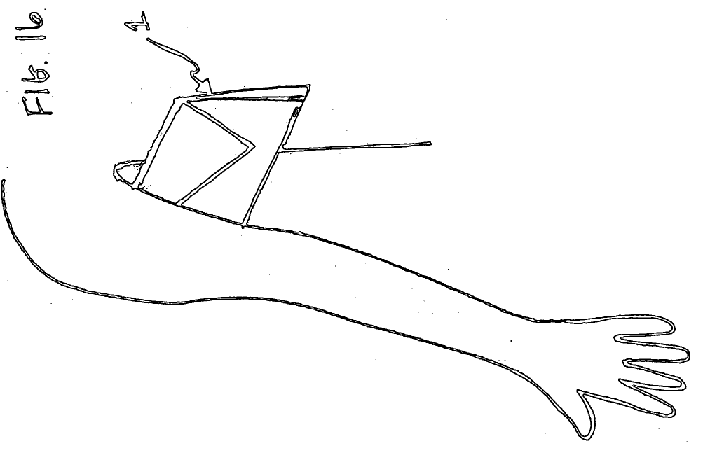

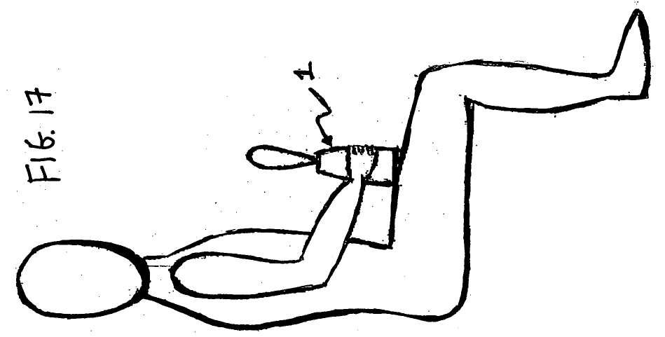

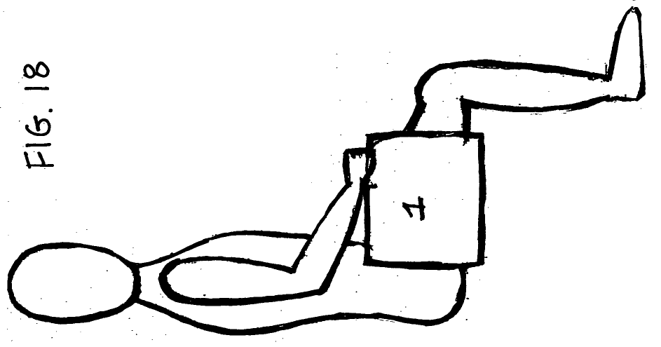

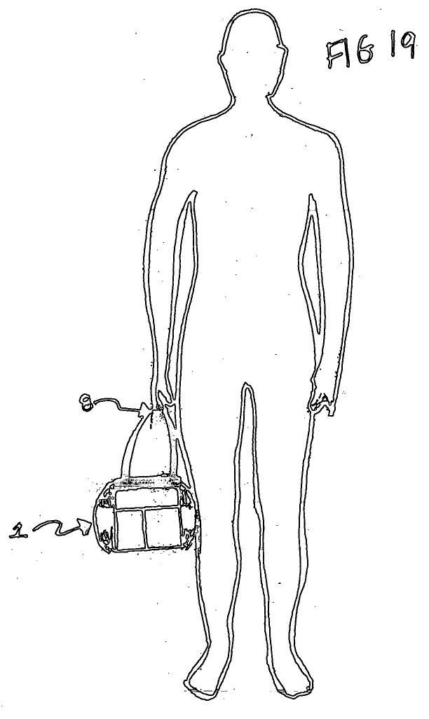

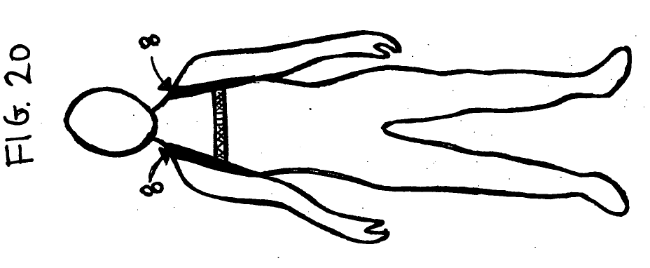

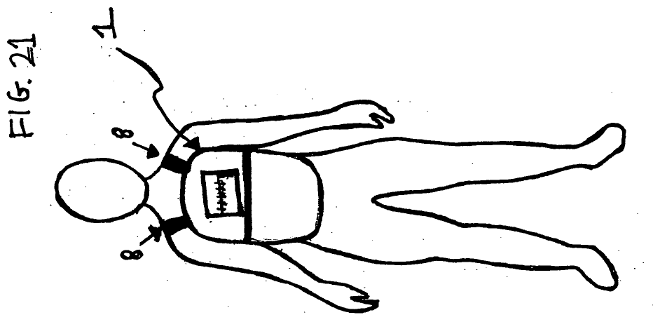

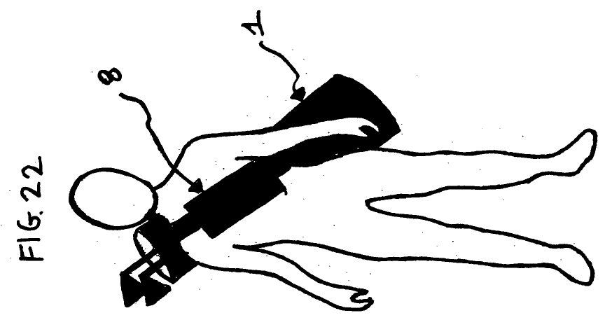

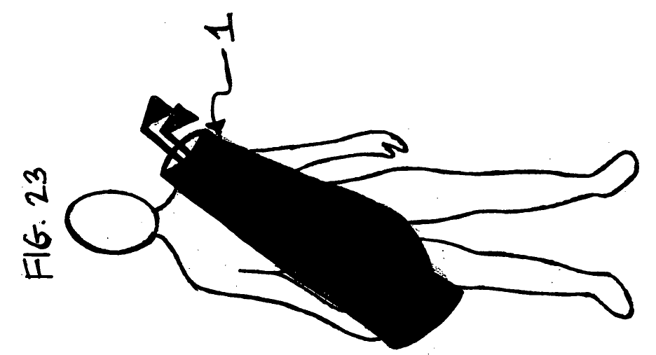

FIGS. 9-23 show various embodiments of the present invention. In each of these Figures, a bag 1 and a strap 8 are shown. In each of these alternative embodiments, magnetic devices and/or SRT™ devices can be inserted into a carrying portion of the bag 1, as shown, for example, in FIGS. 4-8, and/or can be included in a strap portion 8 of the bag 1, as shown, for example, in FIGS. 4-8.

It is also possible to include such devices as a animal carrying container such that, for example, small dogs and cats could also benefit from therapeutic fields.

Accordingly, the present invention provides a novel delivery device for delivering therapeutic treatment to help people to cope with the various levels of stress in their daily activities. The present invention would work to decrease stress and provide a level of therapeutic healing. The bags, containing a number of arranged magnetic devices and/or SRT™ devices, could reduce stress, lessen pain and promote and overall health well-being. These therapeutic benefits occur at times when they may be most needed, namely, when stress levels may be highest. For those people who wear a magnetic device throughout the day, the invention would act as a supplement. Those who do not wear a magnetic device can use the present invention intermittently to provide some therapy. A handbag can be worn for many occasions throughout the day. Therefore, one who carries it, will benefit from its magnetic energy without being inconvenienced.

1. A therapeutic bag comprising:
a carrying portion;
a strap portion; and
at least one magnetic device placed in at least one of the carrying portion and the strap portion.

2. The therapeutic bag of claim 1, wherein the strap portion contains at least one magnetic device which occupies at least 25 percent of the length of the strap portion.

3. The therapeutic bag of claim 1, wherein the strap portion contains at least one magnetic device which occupies at least 50 percent of the length of the strap portion.

4. The therapeutic bag of claim 1, wherein the strap portion contains at least one magnetic device which occupies at least 90 percent of the length of the strap portion.

5. The therapeutic bag of claim 1, wherein the strap portion contains at least one magnetic device which occupies at least 25 percent of the length of the strap portion and at least one additional magnetic device is located in said carrying portion.

6. The therapeutic bag of claim 4, wherein said strap portion is capable of being worn by a user diagonally from one shoulder to an opposite side of the torso at the waist.

7. A therapeutic bag comprising:
a carrying portion;
a strap portion; and
at least one sympathetic resonance technology device placed in at least one of the carrying portions and the strap portion.

8. A therapeutic bag comprising:
a carrying portion;
a strap portion; and
at least one magnetic device and at least one sympathetic resonance technology device placed in at least one of the carrying portion and the strap portion.
